(19) United States
(12) Patent Application Publication
(10) Pub. No.: US 2006/0189584 A1
(43) Pub. Date: Aug. 24, 2006

(54) Pharmaceutical combinations

(75) Inventors: John Dixon, Leicestershire (GB); Robert Humphries, Leicestershire (GB); Alexander Nicol, Leicestershire (GB)

Correspondence Address: FISH & NEAVE IP GROUP;ROPES & GRAY LLP, ONE INTERNATIONAL PLACE, BOSTON, MA, 02110-2624, US

(73) Assignee: AstraZeneca AB, Sodertalje (SE)

(21) Appl. No.: 11/406,746

(22) Filed: Apr. 17, 2006

Related U.S. Application Data
(63) Continuation of application No. 10/479,019, filed on Nov. 25, 2003, now abandoned.

(30) Foreign Application Priority Data
May 31, 2001 (SE) 0101932-2

(51) Int. Cl. A61K 31/60; A61K 31/519; A61K 31/4743

(52) U.S. Cl. 514/165; 514/261.1; 514/262.1; 514/301

(57) ABSTRACT

The present invention provides novel pharmaceutical combinations and their use in anti-thrombotic therapy. The combinations comprise a compound of formula (I) or a pharmaceutically acceptable derivative thereof; formula (I), and another anti-thrombotic agent or a pharmaceutically acceptable derivative thereof.

### FIELD OF THE INVENTION

The present invention relates to pharmaceutical combinations comprising a P_{2T} (P2Y₁₂) receptor antagonist and another anti-thrombotic agent and to their use in the treatment and prevention of thrombosis.

### BACKGROUND OF THE INVENTION

Increased understanding of the mechanisms underlying thrombosis and of interventions therein has led to a polypharmacological anti-thrombotic approach utilising anti-platelet, anti-coagulant and fibrinolytic agents in combinations appropriate to either acute treatment or secondary prevention. Examples of anti-thrombotic compounds used include anti-platelet agents such as aspirin, clopidogrel, ticlopidine, dipyridamole, GPIIb/IIIa antagonists; anti-coagulants such as thrombin inhibitors, warfarin, factor Xa inhibitors, heparin and low molecular weight heparins; and fibrinolytic agents including but not limited to, streptokinase, tissue plasminogen activator (tPA) and tenecteplase.

International Patent Application WO 97/29753 discloses a pharmaceutical composition containing clopidogrel and aspirin. International Patent Application WO 00/53264 discloses a method of treating thrombosis by administering a combination of a factor Xa inhibitor and a compound selected from aspirin, tPA, a GPIIb/IIIa antagonist, low molecular weight heparin and heparin. International Patent Application WO 00/64470 discloses a pharmaceutical formulation comprising a low molecular weight thrombin inhibitor and a prodrug of a low molecular weight thrombin inhibitor.

Although progress has been made, a remaining shortcoming of existing anti-thrombotic agents, and combinations thereof, is that the optimal pharmacodynamic risk:benefit (anti-thrombotic:anti-haemostatic) relationship has not yet been achieved. Thus there is a need for more effective anti-thrombotic therapy.

International Patent Application WO 9905143 discloses generically a series of triazolo[4,5-d]pyrimidine compounds having activity as P_{2T} (also known as P2Y₁₂, P2Y_{ADP} or P2T_{AC}) antagonists. Recently, a new class of direct (that is non-prodrug) P_{2T} receptor antagonists has been described which offers significant improvements over other anti-thrombotic agents. International Patent Application WO 0034283 discloses novel “direct” P_{2T} receptor antagonists, including compounds of formula (I) (see below). These compounds may be used in any condition where platelet activation or aggregation is involved. The compounds may thus act as anti-thrombotic agents and may be used in primary and secondary prevention and treatment of thrombotic complications

### DISCLOSURE OF THE INVENTION

The inventors of the present invention have surprisingly found that administration of compound of formula (I): 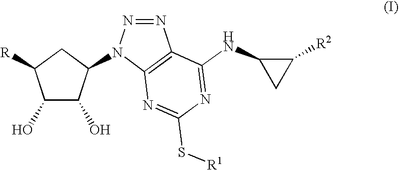
wherein:
R is CH₂OH or O(CH₂)₂OH;
R¹ is C₃₋₄ alkyl optionally substituted by three halogen atoms;
R² is phenyl or 3,4-difluorophenyl;
or a pharmaceutically acceptable derivative thereof,
and another anti-thrombotic agent or a pharmaceutically acceptable derivative thereof, offers a significant improvement over other currently available combination anti-thrombotic treatments.

Accordingly, the combined administration of the compound of formula (I) or a pharmaceutically acceptable derivative thereof and another anti-thrombotic agent or a pharmaceutically acceptable derivative thereof, can be used in the treatment and prevention of thrombosis, particularly in the treatment of the thrombotic complications of atherosclerotic disease and interventions therein.

According to a first aspect of the invention there is provided a kit of parts comprising:

(a) a compound of formula (I) or a pharmaceutically acceptable derivative thereof (component a); and

(b) another anti-thrombotic agent or a pharmaceutically acceptable derivative thereof (component b);

where components (a) and (b) are each provided in a form (which may be the same or different) that is suitable for administration in conjunction with each other.

Pharmaceutically acceptable derivatives of a compound of formula (I) and other anti-thrombotic agent include salts (e.g. pharmaceutically acceptable non-toxic organic or inorganic acid addition salts (such as a salt of hydrochloric, hydrobromic, nitric, sulphuric or acetic acid)), solvates and solvates of salts.

If more than one formulation comprising a compound of formula (I) or another anti-thrombotic agent is present, for example in order to provide for repeat dosing, such formulations may be the same, or may be different in terms of the dosage, chemical composition and/or physical form.

Preferably R¹ is n-propyl, 3,3,3-trifluoropropyl or n-butyl.

Preferably the other anti-thrombotic agent is selected from anti-platelet agents, anti-coagulant agents, fibrinolytic agents, and any combination thereof.

More preferably the other anti-thrombotic agent is selected from the group consisting of but not limited to aspirin, clopidogrel, ticlopidine, dipyridamole, a GPIIb/IIIa antagonist, a direct thrombin inhibitor, a prodrug of a direct thrombin inhibitor, warfarin, a factor Xa inhibitor, heparin, a low molecular weight heparin, tissue plasminogen activator, tenecteplase, or any combination thereof.

Suitable examples of a direct thrombin inhibitor include melagatran (WO 94/29336). Suitable examples of a prodrug of a direct thrombin inhibitor include those described in WO 97/23499, and particularly include Example 17 of that application. Example 17 of WO 97/23499 is H 376/95, which is EtO₂C—CH₂—(R)Cgl-Aze-Pab-OH, wherein Cgl is cyclohexylglycinyl, Aze is (S)-azetidine-2-carbonyl and Pab is para-amidinobenzylamino and the OH replaces one of the amidino hydrogens in Pab.

In accordance with the invention, the compound of formula (I), other anti-thrombotic agent, and derivatives of either, may be administered orally, intravenously, subcutaneously, buccally, rectally, dermally, nasally, tracheally, bronchially, topically, or via inhalation into the lung. Preferred modes of delivery are systemic. For the compound of formula (I) and derivatives thereof, preferred modes of administration are oral. For the other anti-thrombotic agent and derivatives thereof, preferred modes of administration are oral or, in the case of unfractionated or low molecular weight heparins, certain direct thrombin inhibitors and fibrinolytic agents, intravenous or subcutaneous.

The sequence in which the formulations comprising the compound of formula (I) and the other anti-thrombotic agent may be administered (i.e. whether, and at what point, sequential, separate and/or simultaneous administration takes place) may be determined by the physician or skilled person. For example, the sequence may depend upon many factors, such as whether, at any time during the course or period of treatment, one or other of the formulations cannot be administered to the person for practical reasons (e.g. the person is unconscious and thus unable to take an oral formulation).

Respective formulations comprising the compound of formula (I) and/or other anti-thrombotic agent may be administered, sequentially, separately and/or simultaneously, over the course of treating the relevant condition, which condition may be acute or chronic.

Preferably the two formulations are administered (optionally repeatedly) sufficiently closely in time for there to be a beneficial effect for the patient, that is greater, over the course of the treating the relevant condition, than if either of the two formulations are administered (optionally repeatedly) alone, in the absence of the other formulation, over the same course of treatment. Determination of whether a combination provides a greater beneficial effect in respect of, and over the course of treatment of a particular condition, will depend upon the condition to be treated or prevented, but may be achieved routinely by the skilled person.

Alternatively, one or other of the two component formulations may be administered (optionally repeatedly) prior to, after, and/or at the same time as, administration with the other component. Individual doses of a compound of formula (I) and other anti-thrombotic agent may be used within 48 hours (e.g. 24 hours) of each other.

In the therapeutic treatment of mammals, and especially humans, the compound of formula (I), other anti-thrombotic agent, and derivatives of either, may be administered alone, but will generally be administered as a pharmaceutical formulation in admixture with a pharmaceutically acceptable adjuvant, diluent or carrier, which should be selected with due regard to the intended route of administration and standard pharmaceutical practice.

In accordance with the invention, the kit of parts may be used in medical therapy, suitably in the treatment of thrombosis. The treatment of thrombosis will be understood by those skilled in the art to include the treatment and prevention of thrombotic complications of atherosclerotic disease and interventions therein, such as fibrinolysis, endarterectomy or percutaneous transluminal coronary revascularisation (PTCR), including, but not limited to, percutaneous transluminal coronary angioplasty (PTCA) with or without stenting. Thrombotic complications of atherosclerotic disease include, but are not limited to, acute coronary syndrome (encompassing acute myocardial infarction with or without ST elevation and unstable angina) and thrombotic stroke.

A further aspect of the invention provides a method of treating thrombosis (for example thrombotic complications of atherosclerotic disease and interventions therein, such as fibrinolysis, endarterectomy or percutaneous transluminal coronary revascularisation (PTCR), including, but not limited to, percutaneous transluminal coronary angioplasty (PTCA) with or without stenting) which comprises using a kit of parts for administering a therapeutically effective amount of a P_{2T} receptor and another anti-thrombotic agent to a person suffering from or susceptible to such a disorder.

For avoidance of doubt the term “treatment” includes therapeutic and/or prophylactic treatment.

According to another aspect of the invention, there is provided a method of making a kit of parts as defined herein, which comprises bringing a compound of formula (I) into association with a another anti-thrombotic agent thus rendering the two components suitable for administration in conjunction with each other. By bringing the two components into association with each other, we include that the compound of formula (I) and the other anti-thrombotic agent may be:

i) packaged presented and purchased as separate formulations which are subsequently used in conjunction in combination therapy; or

ii) packaged and presented together as separate components of a combination pack for use in conjunction with each other in combination therapy.

The present invention still further provides a kit of parts comprising:

(1) the compound of formula (I) and other anti-thrombotic agent as defined herein; together with

(2) instructions to use the components in conjunction with each other.

The invention further provides the use of a compound of formula (I), or a pharmaceutically acceptable derivative thereof, in the manufacture of a kit of parts for the treatment of thrombosis.

The compound of formula (I) and other anti-thrombotic agent as described herein may also be co-formulated as a combined preparation (i.e. presented as a single formulation including a compound of formula (I) and other anti-thrombotic agent).

Thus, a further aspect of the invention provides a pharmaceutical formulation comprising:

(a) a compound of formula (I) or a pharmaceutically acceptable derivative thereof; and

(b) another anti-thrombotic agent or a pharmaceutically acceptable derivative thereof; in admixture with a pharmaceutically acceptable adjuvant, diluent or carrier.

Preferably R¹ is n-propyl, 3,3,3-trifluoropropyl or n-butyl.

Preferably the other anti-thrombotic agent is selected from anti-platelet agents, anti-coagulant agents, fibrinolytic agents, and any combination thereof.

More preferably the other anti-thrombotic agent is selected from the group consisting of but not limited to aspirin, clopidogrel, ticlopidine, dipyridamole, a GPIIb/IIIa antagonist, a direct thrombin inhibitor, a prodrug of a direct thrombin inhibitor, warfarin, a factor Xa inhibitor, heparin, a low molecular weight heparin, tissue plasminogen activator, tenecteplase, or any combination thereof.

Suitable examples of a direct thrombin inhibitor include melagatran (WO 94/29336). Suitable examples of a prodrug of a direct thrombin inhibitor include EtO₂C—CH₂—(R)Cgl-Aze-Pab-OH (WO 97/23499).

The present invention provides a pharmaceutical formulation comprising:

(a) a compound of formula (I) or a pharmaceutically acceptable derivative thereof; and

(b) another anti-thrombotic agent or a pharmaceutically acceptable derivative thereof, in admixture with a pharmaceutically acceptable adjuvant, diluent or carrier;

for use in medical therapy, suitably in the treatment of thrombosis.

The invention further provides a method of treating thrombosis which comprises administering a therapeutically effective amount of a pharmaceutical formulation comprising:

(a) a compound of formula (I) or a pharmaceutically acceptable derivative thereof; and

(b) another anti-thrombotic agent or a pharmaceutically acceptable derivative thereof, in admixture with a pharmaceutically acceptable adjuvant, diluent or carrier;

to a person suffering from or susceptible to such a disorder.

In another aspect of the present invention, there is provided a process for the preparation of a pharmaceutical formulation which comprises mixing a compound of formula (I) with another anti-thrombotic agent.

The invention further provides the use of a pharmaceutical formulation as defined above in the manufacture of a medicament for the treatment of thrombosis.

Another aspect of the invention involves the use of:

(a) a pharmaceutical formulation comprising a compound of formula (I) or a pharmaceutically acceptable derivative thereof, in admixture with a pharmaceutically acceptable adjuvant, diluent or carrier, and

(b) a pharmaceutical formulation comprising another anti-thrombotic agent or a pharmaceutically acceptable derivative thereof, in admixture with a pharmaceutically acceptable adjuvant, diluent or carrier,

in therapy, suitably in the treatment of thrombosis.

A further aspect of the invention provides a method of treating thrombosis which comprises administering:

a) a pharmaceutical formulation comprising a compound of formula (I) or a pharmaceutically acceptable derivative thereof, in admixture with a pharmaceutically acceptable adjuvant, diluent or carrier, and

b) a pharmaceutical formulation comprising another anti-thrombotic agent or a pharmaceutically acceptable derivative thereof, in admixture with a pharmaceutically acceptable adjuvant, diluent or carrier,

to a person suffering from or susceptible to such a disorder.

Preferably R¹ is n-propyl, 3,3,3-trifluoropropyl or n-butyl.

Preferably the other anti-thrombotic agent is selected from anti-platelet agents, anti-coagulant agents, fibrinolytic agents, and any combination thereof.

More preferably the other anti-thrombotic agent is selected from the group consisting of but not limited aspirin, clopidogrel, ticlopidine, dipyridamole, a GPIIb/IIIa antagonist, a direct thrombin inhibitor, a prodrug of a direct thrombin inhibitor, warfarin, a factor Xa inhibitor, heparin, a low molecular weight heparin, tissue plasminogen activator, tenecteplase, or any combination thereof.

Suitable examples of a direct thrombin inhibitor include melagatran (WO 94/29336). Suitable examples of a prodrug of a direct thrombin inhibitor include EtO₂C—CH₂—(R)Cgl-Aze-Pab-OH (WO 97/23499).

In another aspect of the present invention, there is provided the use of a compound of formula (I), or a pharmaceutically acceptable derivative thereof, in the manufacture of a medicament to be used in combination with another anti-thrombotic agent in the treatment of thrombosis.

Suitable formulations for administering a compound of formula (I) are known in the art, and include those known from WO0034283

Suitable formulations for administering other anti-thrombotic agent are described in the literature, for example, when the other anti-thrombotic agent is melagatran, or a prodrug of melagatran, suitable formulations include those described in inter alia WO 94/29336, WO 96/14084, WO 96/16671, WO 97/23499, WO 97/39770, WO 97/45138, WO 98/16252, WO 99/27912, WO 99/27913, WO 00/13672 and WO 00/12043. Otherwise, the preparation of suitable formulations may be achieved by the skilled person using routine techniques.

Suitable doses of the compound of formula (I), the other anti-thrombotic agent, and derivatives of either can be determined by the medical practitioner or other skilled person, and will depend on the severity of the condition, and on the person to be treated, as well as the compound(s) which is/are employed. Respective doses are discussed in the prior art documents disclosing compounds of formula (I) and other anti-thrombotic agents that are mentioned above.

In the case of a compound of formula (I), suitable doses of active compound in the therapeutic and/or prophylactic treatment of mammalian, especially human, patients include those which give a mean plasma concentration of up to 10 μmol/L, for example in the range 0.001 to 10 μmol/L over the course of treatment of the relevant condition. In any event, the physician, or the skilled person, will be able to determine the actual dosage which will be most suitable for an individual person, which is likely to vary with the condition that is to be treated, as well as the age, weight, sex and response of the particular person to be treated. The above-mentioned dosages are exemplary of the average case. There can, of course, be individual instances where higher or lower dosage ranges are merited, and such are within the scope of this invention.

The pharmaceutical formulation of the invention may, and indeed will usually, contain various other ingredients known in the art, for example preservatives, stabilising agents, viscosity-regulating agents, emulsifying agents or buffering agents. Thus the pharmaceutical formulation of the invention will typically comprise a total amount of (a) the compound of formula (I) and (b) another anti-thrombotic agent (the active ingredients) in the range from 0.05 to 99% w (percent by weight), more preferably in the range from 0.10 to 70% w, and even more preferably in the range from 0.10 to 50% w, all percentages by weight being based on total formulation.

According to a further aspect of the invention there is provided a compound of formula (I) which is compound (A): 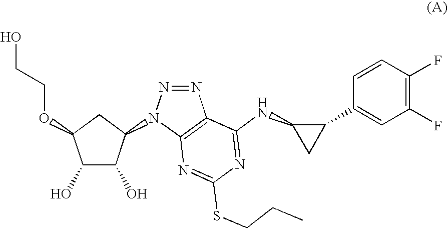
in combination with aspirin, clopidogrel, ticlopidine, dipyridamole, a GPIIb/IIIa antagonist, a direct thrombin inhibitor, a prodrug of a direct thrombin inhibitor, warfarin, a factor Xa inhibitor, heparin, a low molecular weight heparin, tissue plasminogen activator, tenecteplase, or any combination thereof.

According to another aspect of the invention there is provided a compound of formula (I) which is compound (B): 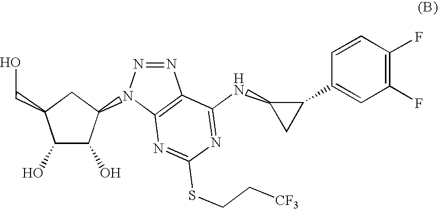
in combination with aspirin, clopidogrel, ticlopidine, dipyridamole, a GPIIb/IIIa antagonist, a direct thrombin inhibitor, a prodrug of a direct thrombin inhibitor, warfarin, a factor Xa inhibitor, heparin, a low molecular weight heparin, tissue plasminogen activator, tenecteplase, or any combination thereof

According to a further aspect of the invention there is provided a compound of formula (I) which is compound (C): 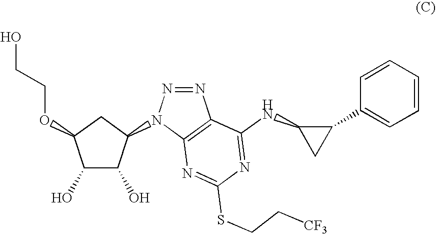
in combination with aspirin, clopidogrel, ticlopidine, dipyridamole, a GPIIb/IIIa antagonist, a direct thrombin inhibitor, a prodrug of a direct thrombin inhibitor, warfarin, a factor Xa inhibitor, heparin, a low molecular weight heparin, tissue plasminogen activator, tenecteplase, or any combination thereof.

According to the invention there is further provided a compound of formula (I) which is compound (D): 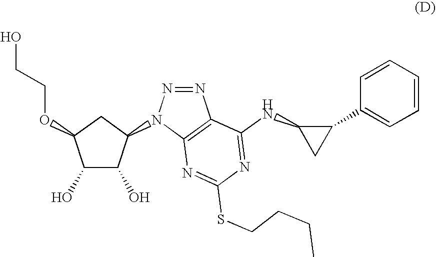
in combination with aspirin, clopidogrel, ticlopidine, dipyridamole, a GPIIb/IIIa antagonist, a direct thrombin inhibitor, a prodrug of a direct thrombin inhibitor, warfarin, a factor Xa inhibitor, heparin, a low molecular weight heparin, tissue plasminogen activator, tenecteplase, or any combination thereof.

### EXAMPLES

The invention is illustrated but in no way limited by the following example.

### Example 1

Canine Femoral Artery Thrombosis Model—Compound A and Aspirin

Compound A as defined above was used in combination with aspirin in a dog model of femoral artery thrombosis to determine whether combination of a P_{2T}-receptor antagonist and pre-treatment with aspirin would have an improved profile when compared to the effect of either agent used alone.

The results of the experiments are evident in FIG. 1, in which there is a clear (though not statistically-significant) trend for an increased anti-thrombotic potency (as assessed by the dose (ID₅₀) required to produce 50% inhibition of thrombosis) of Compound A when administered in combination with aspirin.

[image]

Abbreviations

ADP=adenosine diphosphate

GPIIb/IIIa antagonist=glycoprotein IIb/IIIa antagonist

PTCR=percutaneous transluminal coronary revascularisation

PTCA=percutaneous transluminal coronary angioplasty

1. A kit of parts comprising:
(a) a compound of formula (I) 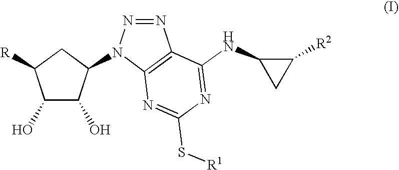
wherein:
R is CH₂OH or O(CH₂)₂OH;
R¹ is C₃₋₄ alkyl optionally substituted by three halogen atoms;
R² is phenyl or 3,4-difluorophenyl;
or a pharmaceutically acceptable derivative thereof, (component a); and
(b) another anti-thrombotic agent or a pharmaceutically acceptable derivative thereof (component b);
where components (a) and (b) are each provided in a form (which may be the same or different) that is suitable for administration in conjunction with each other.

2. A kit of parts according to claim 1 wherein R¹ is n-propyl, 3,3,3-trifluoropropyl or n-butyl.

3. A kit of parts according to claim 1 or 2, wherein the anti-thrombotic agent is selected from anti-platelet agents, anti-coagulant agents, fibrinolytic agents, and any combination thereof.

4. A kit of parts according to any one of claims 1 to 3, wherein the anti-thrombotic agent is selected from the group consisting of aspirin, clopidogrel, ticlopidine, dipyridamole, a GPIIb/IIIa antagonist, a direct thrombin inhibitor, a prodrug of a direct thrombin inhibitor, warfarin, a factor Xa inhibitor, heparin, a low molecular weight heparin, tissue plasminogen activator, tenecteplase, or any combination thereof.

5. A kit of parts according to any one of claims 1 to 4, wherein the anti-thrombotic agent is a direct thrombin inhibitor and/or a prodrug of a direct thrombin inhibitor.

6. A kit of parts as claimed in claim 5 wherein the thrombin inhibitor is melagatran.

7. A kit of parts as claimed in claim 5 wherein the prodrug of a direct thrombin inhibitor is EtO₂C—CH₂—(R)Cgl-Aze-Pab-OH.

8. A kit of parts according to any one of claims 1 to 7, wherein components (a) and (b) are suitable for sequential, separate and/or simultaneous administration.

9. A kit of parts according to any one of claims 1 to 7, for use in medical therapy.

10. A kit of parts according to any one of claims 1 to 7, for use in the treatment of thrombosis.

11. A method of treating thrombosis which comprises using a kit of parts according to any one of claims 1 to 7, for administering a therapeutically effective amount of a P_{2T} receptor and another anti-thrombotic agent to a person suffering from or susceptible to such a disorder.

12. The use of a compound of formula (I) according to any one of claims 1 to 11, or a pharmaceutically acceptable derivative thereof, in the manufacture of a kit of parts for the treatment of thrombosis.

13. A pharmaceutical formulation comprising:
(a) a compound of formula (I) or a pharmaceutically acceptable derivative thereof; and
(b) another anti-thrombotic agent or a pharmaceutically acceptable derivative thereof;
in admixture with a pharmaceutically acceptable adjuvant, diluent or carrier.

14. A pharmaceutical formulation according to claim 13 wherein R¹ is n-propyl, 3,3,3-trifluoropropyl or n-butyl.

15. A pharmaceutical formulation according to claim 13 or 14, wherein the anti-thrombotic agent is selected from anti-platelet agents, anti-coagulant agents, fibrinolytic agents, and any combination thereof.

16. A pharmaceutical formulation according to any one of claims 13 to 15, wherein the anti-thrombotic agent is selected from the group consisting of aspirin, clopidogrel, ticlopidine, dipyridamole, a GPIIb/IIIa antagonist, a direct thrombin inhibitor, a prodrug of a direct thrombin inhibitor, warfarin, a factor Xa inhibitor, heparin, a low molecular weight heparin, tissue plasminogen activator, tenecteplase, or any combination thereof.

17. A pharmaceutical formulation according to any one of claims 13 to 16, wherein the anti-thrombotic agent is a direct thrombin inhibitor and/or a prodrug of a direct thrombin inhibitor.

18. A pharmaceutical formulation according to claim 17 wherein the thrombin inhibitor is melagatran.

19. A pharmaceutical formulation according to claim 17 wherein the prodrug of a direct thrombin inhibitor is EtO₂C—CH₂—(R)Cgl-Aze-Pab-OH.

20. A pharmaceutical formulation according to any one of claims 13 to 19, for use in medical therapy.

21. A pharmaceutical formulation according to any one of claims 13 to 19, for use in the treatment of thrombosis.

22. The use of a pharmaceutical formulation according to any one of claims 13 to 19, in the manufacture of a medicament for the treatment of thrombosis.

23. A method of treating thrombosis which comprises administering a therapeutically effective amount of a pharmaceutical formulation according to any one of claims 13 to 19, to a person suffering from or susceptible to such a disorder.

24. A process for the preparation of a pharmaceutical formulation according to any one of claims 13 to 19, which comprises mixing a compound of formula (I) with another anti-thrombotic agent.

25. The use of:
(a) a pharmaceutical formulation comprising a compound of formula (I) or a pharmaceutically acceptable derivative thereof, in admixture with a pharmaceutically acceptable adjuvant, diluent or carrier; and
(b) a pharmaceutical formulation comprising another anti-thrombotic agent or a pharmaceutically acceptable derivative thereof, in admixture with a pharmaceutically acceptable adjuvant, diluent or carrier,
in therapy.

26. The use of:
(a) a pharmaceutical formulation comprising a compound of formula (I) or a pharmaceutically acceptable derivative thereof, in admixture with a pharmaceutically acceptable adjuvant, diluent or carrier; and
(b) a pharmaceutical formulation comprising another anti-thrombotic agent or a pharmaceutically acceptable derivative thereof, in admixture with a pharmaceutically acceptable adjuvant, diluent or carrier,
in the treatment of thrombosis

27. A method of treating thrombosis which comprises administering to a person suffering from, or susceptible to such a condition:
(a) a pharmaceutical formulation comprising a compound of formula (I), or a pharmaceutically acceptable derivative thereof, in admixture with a pharmaceutically acceptable adjuvant, diluent or carrier, and
(b) a pharmaceutical formulation comprising another anti-thrombotic agent or a pharmaceutically acceptable derivative thereof, in admixture with a pharmaceutically acceptable adjuvant, diluent or carrier.

28. A method according to claim 27 wherein R¹ is n-propyl, 3,3,3-trifluoropropyl or n-butyl.

29. A method according to claim 27 or 28, wherein the anti-thrombotic agent is selected from anti-platelet agents, anti-coagulant agents, and any combination thereof.

30. A method according to any one of claims 27 to 29, wherein the anti-thrombotic agent is selected from the group consisting of aspirin, clopidogrel, ticlopidine, dipyridamole, a GPIIb/IIIa antagonist, a direct thrombin inhibitor, a prodrug of a direct thrombin inhibitor, warfarin, a factor Xa inhibitor, heparin, a low molecular weight heparin, tissue plasminogen activator, tenecteplase, or any combination thereof

31. A method according to any one of claims 27 to 30, wherein the anti-thrombotic agent is a direct thrombin inhibitor and/or a prodrug of a direct thrombin inhibitor.

32. A method according to claim 31 wherein the thrombin inhibitor is melagatran.

33. A method according to claim 31 wherein the prodrug of a direct thrombin inhibitor is EtO₂C—CH₂—(R)Cgl-Aze-Pab-OH.

34. The use of a compound of formula (I) as defined in claim 1, or a pharmaceutically acceptable derivative thereof, in the manufacture of a medicament to be used in combination with another anti-thrombotic agent in the treatment of thrombosis.

35. A compound of formula (I) which is: 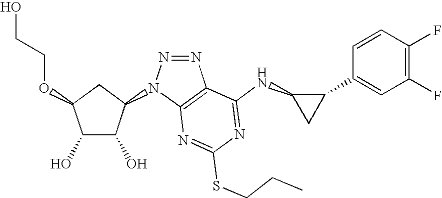
in combination with aspirin, clopidogrel, ticlopidine, dipyridamole, a GPIIb/IIIa antagonist, a direct thrombin inhibitor, a prodrug of a direct thrombin inhibitor, warfarin, a factor Xa inhibitor, heparin, a low molecular weight heparin, tissue plasminogen activator, tenecteplase, or any combination thereof

36. A compound of formula (I) which is: 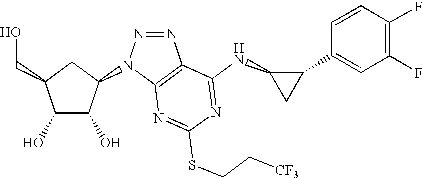
in combination with aspirin, clopidogrel, ticlopidine, dipyridamole, a GPIIb/IIIa antagonist, a direct thrombin inhibitor, a prodrug of a direct thrombin inhibitor, warfarin, a factor Xa inhibitor, heparin, a low molecular weight heparin, tissue plasminogen activator, tenecteplase, or any combination thereof.

37. A compound of formula (I) which is: 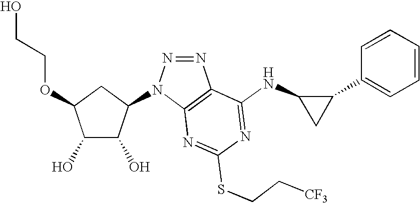
in combination with aspirin, clopidogrel, ticlopidine, dipyridamole, a GPIIb/IIIa antagonist, a direct thrombin inhibitor, a prodrug of a direct thrombin inhibitor, warfarin, a factor Xa inhibitor, heparin, a low molecular weight heparin, tissue plasminogen activator, tenecteplase, or any combination thereof.

38. A compound of formula (I) which is: 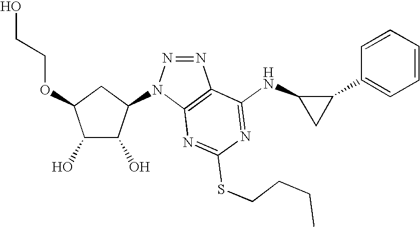
in combination with aspirin, clopidogrel, ticlopidine, dipyridamole, a GPIIb/IIIa antagonist, a direct thrombin inhibitor, a prodrug of a direct thrombin inhibitor, warfarin, a factor Xa inhibitor, heparin, a low molecular weight heparin, tissue plasminogen activator, tenecteplase, or any combination thereof.
